(19) United States
(12) Patent Application Publication
(10) Pub. No.: US 2009/0048430 A1
(43) Pub. Date: Feb. 19, 2009

(54) Site specific protein modification

(75) Inventors: Homme W. Hellinga, Durham, NC (US); James J. Smith, Durham, NC (US); David W. Conrad, Durham, NC (US)

Correspondence Address: NIXON & VANDERHYE, PC, 901 NORTH GLEBE ROAD, 11TH FLOOR, ARLINGTON, VA, 22203, US

(73) Assignee: DUKE UNIVERSITY, Durham, NC (US)

(21) Appl. No.: 11/987,006

(22) Filed: Nov. 26, 2007

Related U.S. Application Data
(63) Continuation of application No. 11/591,675, filed on Nov. 2, 2006, now abandoned.
(60) Provisional application No. 60/732,142, filed on Nov. 2, 2005.
(60) Provisional application No. 60/732,650, filed on Nov. 3, 2005.

(51) Int. Cl. C07K 1/00

(52) U.S. Cl. 530/335

(57) ABSTRACT

The present invention relates, in general, to protein modifications and, in particular, to a method of effecting site-specific labeling of proteins with covalently coupled reporter groups. The invention further relates to a method of effecting orientation-specific immobilization of proteins on a solid surface. The invention also relates to products produced by such methods.

This application claims priority from U.S. Provisional Application No. 60/732,142, filed Nov. 2, 2005 and from U.S. Provisional Application No. 60/732,650, filed Nov. 3, 2005, the entire contents of both applications being incorporated herein by reference.

### TECHNICAL FIELD

The present invention relates, in general, to protein modifications and, in particular, to a method of effecting site-specific labeling of proteins with covalently coupled reporter groups. The invention further relates to a method of effecting orientation-specific immobilization of proteins on a solid surface. The invention also relates to products produced by such methods.

### BACKGROUND

Covalent modification is an important natural (Han and Martinage, Int J Biochem 24: 19-28 (1992), Kukuruzinska and Lennon, Crit Rev Oral Biol Med 9: 415-448 (1998), Johnson, Annu Rev Biochem 73: 355-382 (2004)) and biotechnological (DeSantis and Jones, Curr Opin Biotechnol 10: 324-330 (1999), Qi et al, Chem Rev 101: 3081-3111 (2001)) strategy to introduce new functionalities into proteins. Examples include cofactors for catalysis (Kaiser, Angew Chem Int Ed Engl 27: 913-922 (1988), Tann et al, Curr Opin Chem Biol 5: 696-704 (2001)), the use of fluorophores (Marvin et al, Proc Natl Acad Sci USA 94: 4366-4371 (1997)) or electrochemical (Benson et al, Science 293: 1641-1644 (2001)) groups for detection of ligand binding in biosensors, and immobilization on solid surfaces (Domen et al, J Chromatogr. 510: 293-302 (1990), Willner et al, J Biotechnol 82: 325-355 (2002)). It is frequently necessary to modify the protein site-specifically to optimally combine the conjugated functionality with the intrinsic properties of the protein.

As demands on the functionalities of engineered proteins become more sophisticated, it is often desirable to introduce multiple, covalent modifications involving several different functionalities in a site-specific manner. Strategies to produce proteins with single or multiple non-natural amino acids include total synthesis (Jantz and Berg, J Am Chem Soc 125: 4960-4961 (2003)), semi-synthesis by ligation of synthetic and biologically expressed fragments (Muir et al, Proc Natl Acad Sci USA 95: 6705-6710 (1998), Hofmann et al, Bioorg. Med. Chem. Lett. 11: 3091-3094 (2001), Hofmann and Muir Curr Opin Biotechnol 13: 297-303 (2002)), and in vitro translation using a partially extended genetic code (Zhang et al, Biochemistry 42: 6735-6746 (2003), Zhang et al, Proc Natl Acad Sci USA 101: 8882-8887 (2004)). Nevertheless, one of the simplest methods still remains covalent modification of biologically expressed proteins (Hermanson, Bioconjugate Techniques, 1 ed. Academic Press, San Diego, pp. 148 (1996)). This strategy requires a single, uniquely reactive amino acid. Cysteine is well suited for this purpose, since it is relatively rare, and the thiol(ate) presents a uniquely reactive functional group that is readily modified under mild conditions (Hermanson, Bioconjugate Techniques, 1 ed. Academic Press, San Diego, pp. 148 (1996)). Multiple, independent site-specific modifications require more than one differentially reactive cysteine. In rare cases these occur in naturally evolved proteins, permitting different labels to be introduced independently (Tanaka et al, Biochim Biophys Acta 1339: 226-232 (1997)). Engineered cysteine pairs have also been used (Ha et al, Proc Natl Acad Sci USA 96: 893-898 (1999), Ratner et al, Bioconjug Chem 13: 1163-1170 (2002), Schuler et al, Nature 419: 743-747 (2002), Rhoades et al, Proc Natl Acad Sci USA 100: 3197-3202 (2003), Allen et al, Anal Biochem 325: 273-284 (2004)), but typically have insufficient differential reactivity to obtain highly specific double labeling and require additional purification steps to separate the various labeled contaminants.

The present invention provides, at least in part, a method of engineering proteins with multiple, differentially reactive cysteines that are independently addressable through reversible thiol protection (RTP) mechanisms.

### SUMMARY OF THE INVENTION

The present invention relates generally to protein modifications. More specifically, the invention relates to a method of effecting site-specific labeling of proteins with covalently coupled reporter groups. The invention further relates to a method of effecting orientation-specific immobilization of proteins on a solid surface. The invention also relates to products produced by such methods.

Objects and advantages of the present invention will be clear from the description that follows.

### BRIEF DESCRIPTION OF THE DRAWINGS

FIG. 1. Schemes for producing multiple, site-specific modifications in zinc finger fusion proteins using either reversible metal coordination or disulfide mediated protection strategies. Two distinct thiol reactive modifications are represented as [symbol] and ▴.

FIGS. 2A-2C. Analysis of labeling patterns in MBP_{141C}(Cy5)::th::ZifQNK(TMR)₂ and MBP_{141C}(TMR)::th::ZifQNK(Cy5)₂ as indicated (FIG. 2A) Absorbance spectra of doubly labeled proteins. Spectra of the conjugate produced by metal-mediated protection shown at half the concentration of those produced by the disulfide-mediated scheme. Calculated ratios for MBP_{141C}(Cy5)::th::ZifQNK(TMR)₂ with disulfide protection are Cy5/protein=1.09 and TMR/Cy5=2.05 and with metal protection are Cy5/protein=1.07 and TMR/Cy5=2.18. Ratios for MBP_{141C}(TMR)::th::ZifQNK(Cy5)₂ with disulfide protection are TMR/protein=0.97 and TMR/Cy5=0.57 and with metal protection are TMR/protein=0.98 and TMR/Cy5=0.52. (FIG. 2B) HPLC chromatographs of thrombin cleaved MBP_{141C}(Cy5)::th::ZifQNK(TMR)₂ and MBP_{141C}(TMR)::th::ZifQNK(Cy5)₂ produced by the disulfide-mediated scheme. Metal-mediated multiple labeling scheme have identical chromatographs. The three chromatographs represent the same HPLC run monitored at different wavelengths: 280 nm for peptide, 525 nm for TMR, and 650 nm for Cy5. The triple peaks that elute around 10 minutes are the Zif peptides and the single peak at 23 minutes is the MBP peptide. (FIG. 2C) Mass spectra of the doubly-labeled MBP₁₄₁::th::ZifQNK proteins.

FIGS. 3A and 3B. Intramolecular FRET between TMR and Cy5 of MBP_{141C}(Cy5)::th::ZifQNK(TMR)₂ and MBP_{141C}(TMR)::th::ZifQNK(Cy5)₂. (FIG. 3A) Emission spectra obtained in the presence (dashed line) and absence (solid line) of maltose (excitation at 540 nm). Spectra at intermediate maltose concentrations are shown for MBP_{141C}(TMR)::th::ZifQNK(Cy5)₂. Note the presence of an isosbestic point. (FIG. 3B) Titration curves of maltose binding reported as change in the ratio of the summed emission intensities of the donor (560-640 nm) and acceptor (642-700 nm) fluorophores. The measured K_{d} values are 0.2 μM and 2 μM, respectively.

FIGS. 4A and 4B. Preparation and analysis of triply labeled MBP conjugate. (FIG. 4A) Absorbance spectra of double-labeled intermediate, βZif(IAF)₂::th::MBP_{141C}(Cy5)::th::ZifQNK, (dashed line) [Cy5/protein ratio=1.06 and IAF/Cy5 ratio=1.82] and triple-labeled final product, βZif(IAF)₂::th::MBP_{141C}(Cy5)::th::ZifQNK(TMR)₂. (FIG. 4B) Emission intensity spectrum demonstrating the FRET relay effect (exciting IAF at 490 nm). Emission from IAF is observed at 525 nm, TMR at 580 nm, and Cy5 at 670 nm. The apo form is indicated by a solid line and the maltose saturated form is indicated by a dashed line.

FIGS. 5A and 5B. Confocal microscopy images of GBP_{149C}(Cy5)::ZifQNK covalently patterned on BMOE modified glass slides (FIG. 5A) and GBP_{149C}(Cy5) non-specifically absorbed on BMOE modified glass slides (FIG. 5B). Light-grey correspond to Cy5 fluorescence and indicate surface-bound protein. The grid bars are where BMOE was protected from photooxidation by the copper mask. The square pits are areas that were photooxidized.

### DETAILED DESCRIPTION OF THE INVENTION

The present invention relates to a method of producing a fusion protein that comprises multiple covalent modifications that can involve several different functionalities, and to fusion proteins so produced. The invention further relates to kits suitable for use in the instant method.

The present method comprises constructing (e.g., chemically or recombinantly) a fusion protein comprising: i) a protein having properties consistent with the ultimate intended use of the fusion protein fused (C-terminal or N-terminal) to ii) at least one domain (that is, a peptide of about 2 to 1000 amino acids in length, preferably, about 10 to 200 amino acids in length, more preferably, about 20 to about 30 amino acids in length). The protein (i) can comprise, naturally or as a result of engineering, at least a single unprotected and uniquely reactive amino acid. The domain(s) (ii) can comprise one or more uniquely protected reactive amino acids. In accordance with the invention, the unprotected amino acid of protein (i) is reacted with a first reporter group (or other modifying agent (e.g., a co-factor, including, but not limited to, an enzyme co-factor or catalytically active co-factor, a stabilizing agent, an agent that prevents aggregation, a linker group, etc)) so that a covalent linkage between the amino acid and the reporter group (modifying agent) is formed. The reactive amino acid(s) of the domain(s) (ii) can then be deprotected and reacted with a second (e.g., different) reporter group (modifying agent) so that a covalent linkage(s) between that/those amino acid(s) and the reporter group (modifying agent) is (are) formed. When the fusion protein comprises more that one domain (ii), protecting groups can be selected so that deprotection can be effected sequentially.

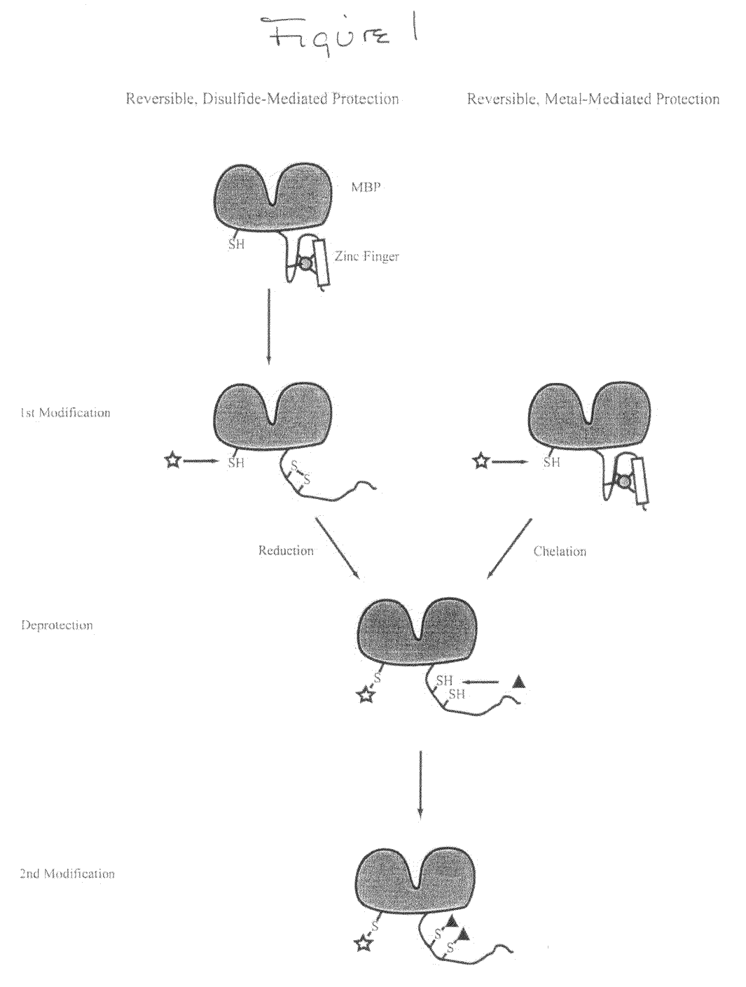

In a preferred embodiment, the reactive amino acids of both the protein (i) and the domain(s) (ii) are, for example, cysteines (including selenocysteines). As shown in FIG. 1, the domain (ii) can be based, for example, on a consensus zinc-finger domain, ZifQNK (Shi and Berg, Science 268: 282-284 (1995)). This 32-residue domain has a Cys₂His₂ primary coordination sphere that binds Zn²⁺ reversibly with 10⁻⁹-10⁻¹¹ M affinity (Michael et al, Proc Natl Acad Sci USA 89: 4796-4800 (1992)). In the absence of Zn²⁺, the two cysteines can form a disulfide under oxidizing conditions (Knapp and Klann, J Biol Chem 275: 24136-24145 (2000)). ZifQNK can, therefore, be used in either metal-dependent or redox-dependent RTP strategies (MRTP, RRTP). Truncated versions of ZifQNK can also be used, an example of a suitable truncated version being one in which the single α-helix bearing the two histidines is deleted, leaving a two-stranded β-sheet containing the two cysteines that readily oxidize to form a disulfide but do not bind Zn²⁺ in the reduced form (designated βZIF). The non-limiting Example that follows describes the use of these domains in the context of fusion proteins constructed with E. coli maltose-binding protein (MBP) that has a single cysteine engineered at position 141 and glucose-binding protein (GBP) that has a single cysteine engineered at position 149.

ZifQNK and βZif fusions are a rapid, straightforward way to add functionalities to almost any protein. The invention, however, is not limited to the use of these domains. Other suitable domains containing disulfides or stable metal centers can be used. Furthermore, the metal-mediated protection scheme can be extended to any thiol protected by a tightly binding ligand. Finally, the approach can be even further generalized by using design methods to introduce disulfides (Ivens et al, Eur. J. Biochem. 269:1145-1153 (2002), Nemeth et al, Biophys. Chem. 96:229-241 (2002)), metal centers (Helling a, Fold Des. 3:R1-8 (1998)), or ligand binding sites (Looger et al, Nature 423:185-190 (2003)) in suitable locations.

The protein (i) component of the fusion protein of the invention can be selected (or engineered) so as to be appropriate for the ultimate intended use of the fusion protein. For example, when use as a biosensor is contemplated, MBP, GBP or other member of the periplasmic binding protein (PBP) superfamily (Tam and Saier, Microbiol Rev 57: 320-346 (1993), de Lorimier et al, Protein Sci 11: 2655-2675 (2002)), can be used. These are soluble, monomeric receptors that consist of two domains linked by a hinge region (Quiocho and Ledvina, Mol Microbiol 20: 17-25 (1996)). The proteins adopt at least two conformations, an open, ligand-free state, and a closed, ligand-bound state, that interconvert upon ligand binding via a hinge-bending motion. Members of the PBP superfamily can be used, for example, to construct reagentless fluorescent and electrochemical sensors by covalently coupling single fluorescent (de Lorimier et al, Protein Sci 11: 2655-2675 (2002)) or redox-active (Benson et al, Science 293: 1641-1644 (2001)) reporter groups, respectively, that respond to the ligand-mediated conformational changes. These motions can also be coupled to changes in fluorescence resonance energy transfer (FRET) between fusions of suitable derivatives of, for example, green fluorescent protein (GFP) at the N- and C-termini of MBP (Fehr et al, Proc Natl Acad Sci USA 99: 9846-9851 (2002)) and other PBPs (Fehr et al, Curr Opin Plant Biol 7: 345-351 (2004)).

The Example below describes the construction of fusion proteins comprising ZifQNK or βZIF at the N- or C-termini of MBP, and demonstrates that these can be used to obtain ligand-responsive FRET between donor and acceptor fluorophores site-specifically coupled at position 141 within MBP (MBP₁₄₁) and the fusion domain. Also described is the construction of a FRET relay (Watrob et al, J Am Chem Soc 125: 7336-7343 (2003)) between three fluorophores in a triply labeled, double fusion protein.

The immobilization of proteins on glass, gold or other non-biological substrates is an important aspect of constructing hybrid devices, such as biosensors (Willner and Katz, Angew Chem Int Ed Engl 39: 1180-1218 (2000), Willner et al, J Biotechnol 82: 325-355 (2002), Willner and Katz, Angew Chem Int Ed Engl 42: 4576-4588 (2003)). It is also an increasingly important component for the construction of protein chips used in genome analysis technologies (Figeys and Pinto, Electrophoresis 22: 208-216 (2001)). Orientation-specific immobilization using defined attachment points on a protein has numerous advantages over random, multipoint chemi- or physisorption (Lu et al, Analyst 121: 29R-32R (1996), Rao et al, Mikrochim. Acta. 128:127-143 (1998), Turkova, J. Chromatogr. B. Biomed. Sci. Appl. 722:11-31 (1999)), especially in cases where binding sites need to be presented, or conformational changes are taken advantage. The site-specific covalent linkage strategies of the present invention offer advantages over non-covalent site-specific linkages, such as provided by a oligohistidine C- or N-terminal fusions (Gershon and Khilko, J. Immunol. Methods 183:65-76 (1995), Allard et al, Biotechnol. Bioeng. 80:341-348 (2002)). As demonstrated in the Example that follows, a fusion protein comprising a protein (i) (e.g., GBP) first labeled with a modifying agent (e.g., a fluorophore) at an unprotected reactive amino acid (e.g., cysteine 149 of GBP) can be patterned on a solid support (e.g., a glass slide) by covalent coupling using reversibly protected reactive amino acids (e.g., cysteines) present in a domain (ii) (e.g., ZifQNK) fused to the protein (i).

As shown in the Example that follows, protection methods can be combined to triple modify proteins and in this case, produce an intramolecular protein FRET relay. FRET relays have utility in overcoming large distances (Watrob et al, J. Am. Chem. Soc. 125:7336-7343 (2003)) and provide large Stokes shifts. Another use for the triple modification strategy can be to immobilize a FRET biosensor to produce a ratiometric device. Different modifications can be combined to immobilize modified proteins (e.g. Cy5 modified protein) in an orientation-specific pattern.

Certain aspects of the invention can be described in greater detail in the non-limiting Example that follows.

### EXAMPLE

### Experimental Details

### Clone Construction

The peptide sequences used for ZifQNK C-terminal and βZif N-terminal fusions with the thrombin cleavage sites were: GLVPR|GSTGEKPYKCPECGKSFSRSDHLSRHQRTHQNKKGSHHHHHH and MTGEKPYKCPECGKSFSRSLVPR|GSGG, respectively (cysteines indicated in bold; linker peptide underlined; thrombin recognition site italicized; cleavage site indicated with |). The C-terminal zinc finger fusion was generated by PCR using the following oligonucleotides: 5′GGAGGTTCAACAGGTGAGAAACCGTACAAGTGCCCGGAGTGTGG CAAATCATTCTCTCGATCGGACCAT, 5′CGGGATCCTATCACTTCTTGTTCTGATGTGTCCGTTGGTGACGGG ATAGATGGTCCGATCGAGAGAATG, and 5′ CTCACCTGTTGAACCTCCCTTGGTCAGCTTAGTCTG. The N-terminal βZif was constructed by PCR using the following oligonucleotides: 5′GGAATTCCATATGACAGGTGAGAAACCGTACAAGTGCCCGGAGT GTGGC and 5′CCTTCTTCGATTTTGCCCCCGGATCCTCGAGGGACGAGCGATCGAG AGAATGATTTGCCACACTCCGGGCA. Wild type MBP was used as template to generate the zinc finger fusions. The MBP A141C mutant was generated by PCR using the following oligonucleotides: 5′GAACTGAATGCAAAGGTAAGAGCGCG and 5′CGCGCTCTTACCTTTGCATTTCAGTTC. All recombinant constructs were cloned into pET21a for expression.

### Protein Expression and Purification

Recombinant proteins were over-expressed in BL21(DE3). 1 L of 2×YT was inoculated with 25 mL from a culture freshly grown to stationary phase (9 h), and grown at 37° C. to an optical density of A₆₀₀=0.4, induced with 1 mM IPTG, and grown for a further 2 h. The cultures were supplemented with 100 μM ZnCl₂ at induction to ensure viability. For MBP fusions, cell pellets were resuspended in IMAC buffer (20 mM MOPS, 500 mM NaCl, 10 mM imidazole; pH 7.5), lysed by sonication (2 min), and a cleared lysate produced by centrifugation (25 min, 25,000×g). The MBP fusions were purified using nickel-charged IMAC resin followed by gel filtration (Superdex 200). Pure protein was quantified by absorbance (ε₂₈₀=66,000 M⁻¹cm⁻¹).

### Labeling Reaction Kinetics

Proteins (1 μM in 50 mM MOPS, 100 mM NaCl; pH 6.0) were reacted with a 5-fold molar excess of CPM (concentrated stock solution in DMSO). The labeling reaction was monitored by following the increase in fluorescence at 470 nm (excitation 385 nm) for the CPM-protein conjugate as a function of time using a fluorescence plate reader (SprectraMAX GeminiXS, Molecular Devices). The values for t_{1/2} were obtained from fits of the data using a commercial software package (TableCurve 2D, SYSTAT Software, Inc.). All experiments were conducted at 25° C.

### Metal-Mediated Reversible Thiol Protection

Proteins were exchanged from purification buffer into modification buffer (50 mM MOPS, 100 mM NaCl; pH 6.0) by gel filtration (Superdex 200). For the first modification (unprotected thiol), 25 μM protein was incubated (room temperature, 30 min; agitated with a roller drum) with 125 μM TCEP, 100 μM ZnCl₂, and 250 μM tetramethylrhodamine 5-maleimide or Cy5 dye in a total volume of 1 mL. The reaction then was transferred to a desalting column (BioRad PD10) pre-equilibrated with modification buffer, collecting the first colored band (modified protein). The labeling efficiency of the first modification was determined as described below. The second pair of thiols were deprotected by chelation in the presence of 5 mM EDTA and 2 mM orthophenathroline (4° C.; 8 h). Following removal of the chelators by gel filtration (Superdex 200), the protein was labeled with 500 μM TMR or Cy5 dye in the presence of 250 μM TCEP, (1-mL reaction volume; 1 h, room temperature; agitated on a roller drum). Unincorporated label was removed by a desalting column (BioRad PD10), eluting with 50 mM MOPS, 100 mM NaCl; pH6.8.

### Redox-Mediated Reversible Thiol Protection

To chelate any free metal, purified protein was first incubated with 5 mM EDTA and 2 mM o-phenanthroline (4° C., 8 h), followed by exchange into 20 mM Tris, 100 mM NaCl; pH 6.0 on a S200 gel filtration column. In these preparations, the disulfide in the ZifQNK peptide was completely oxidized, as determined by DTMB reactivity. For the first modification (unprotected thiol), 25 μM protein was incubated with 250 μM TMR or Cy5 dye (1-mL reaction volume; room temperature for 30 min; agitated on a roller drum). Free fluorophore was removed by desalting column (see above), and the labeling efficiency was determined as described below. Deprotection by reduction and dye modification were carried out in one step by the addition of 250 μM TCEP and 500 μM Cy5 or TMR (1 h at room temperature). Unreacted material was removed by desalting column (see above).

### Triple Modification

The unprotected thiol was labeled first using 25 μM protein and 250 μM Cy5 (30 min at room temperature; agitated on a roller drum). After removing unreacted fluorophore by gel filtration (see above), the βZif domain was deprotected and labeled (125 μM TCEP and 250 μM 5-IAF; 30 min at room temperature). Excess 5-IAF was removed by gel filtration. The ZifQNK domain was deprotected by chelation with 5 mM EDTA and 2 mM o-phenanthroline (8 h at 4° C.), followed by gel filtration and labeling protein with 150 μM TCEP and 250 μM TMR. The triple labeled product was purified from excess fluorophore by gel filtration (see above).

### Determination of Fluorophore Labeling Stoichiometry

Dye-protein ratios were determined using:

$$\frac{D}{P} = \frac{( {A}_{fluor} \times {ɛ}_{protein} )}{( ( {A}_{protein} - ( {A}_{fluor} \times N ) ) \times {ɛ}_{fluor} )}$$

where A_{fluor.} is the absorbance at 650 nm for Cy5 and 525 nm for TMR, Aₚᵣₒₜₑᵢₙ is the absorbance at 280 nm, εₚᵣₒₜₑᵢₙ=66,000 M⁻¹cm⁻¹, ε_{fluor.} is 250,000 M⁻¹cm⁻¹ for Cy5, 95,000 M⁻¹cm⁻¹ for TMR and 75,000 M⁻¹cm⁻¹ for 5-IAF, and N is 0.05 (Amersham Biosciences) for Cy5 and 0.3 for TMR.
The equation for dye/dye ratios was:

$$\frac{D 1}{D 2} = \frac{( {A}_{fluor 1} \times {ɛ}_{fluor 2} )}{( {A}_{fluor 2} \times {ɛ}_{fluor 1} )}$$

where A_{fluor1} is the absorbance for fluorophore 1, A_{fluor2} is the absorbance for fluorophore 2, ε_{fluor1} is the extinction coefficient for fluorophore 1, and ε_{fluor2} is the extinction coefficient for fluorophore 2.

### Thrombin Cleavage and HPLC Purification

Protein was cleaved with biotinylated thrombin according to the manufacturer's protocol (Novagen Thrombin Cleavage Capture Kit). The cleavage products were separated by HPLC (Waters 2795 Alliance HT, PDA detector) using a C4 reversed phase column (Symmetry 300), eluting with a linear gradient from 20% B to 100% B over 80 min at a flow rate of 1 ml/min (A=water with 0.1% TFA; B=acetonitrile with 0.1% TFA). Peaks were identified by absorbance and elution times. Assignments were confirmed by MALDI-TOF mass spectrometry (Applied Biosystems, Voyager DE).

### Fluorescence Spectroscopy

Fluorescence emission intensities were measured at 25° C. in a stirred 1-cm quartz cell using a fluorimeter (AMINCO Bowman Series 2). Protein samples were diluted to 0.2 μM using 20 mM MOPS, 100 mM NaCl; pH 7.0 buffer. Excitation for TMR and IAF was 530 and 490 nm respectively. Fluorescence emission spectra were collected from 550 to 700 nm.

### Protein Immobilization and Confocal Imaging

A glass slide was silanized with a 20:1 ratio of bis(2-hydroxyethyl)-3-aminopropyltriethoxysilane:3-mercaptopropyltrimethoxysilane. A pattern was then produced by photooxidation of the 3-mercaptopropyltrimethoxysilane with short wavelength ultraviolet irradiation for 5 minutes in the presence of a copper mask (10-μm square beehive). Thiols that were protected from photooxidation by the mask were reacted with a homobifunctional crosslinker, bis-maleimidoethane (BMOE). The cysteines in ZifQNK were then deprotected with TCEP, and the GBP₁₄₉(Cy5)::ZifQNK incubated with the slide to react with the maleimide of BMOE. After one hour, the substrate was washed with buffer to remove uncoupled protein, and imaged using a Zeiss LSM-410 confocal microscope.

### Results

Independent double labeling can be achieved using amino- or carboxy-terminal fusions of either ZifQNK or βZIF to protein with a single, unprotected cysteine (FIG. 1). In the case of ZifQNK, either MRTP, or RRTP strategies can be used; for βZIF only RRTP is possible. Independent triple labeling can be achieved using a fusion with both ZifQNK (MRTP) and ZifQNK (RRTP).

### Differential Reactivity of Engineered Thiols

The multiple labeling scheme requires that protected thiols are significantly less reactive than unprotected thiols, and that protection is reversible. To test this, cysteine-free MBP (MBP_{wt}), MBP₁₄₁, MBP_{wt} fused at the C-terminus with ZifQNK in the Zn²⁺ form (MBP_{wt}::ZifQNK•Zn), in the Zn²⁺-free oxidized form (MBP_{wt}::ZifQNKₒₓ), and in the Zn²⁺-free reduced form (MBP_{wt}::ZifQNK_{red}), were reacted with 7-diethylamino-3-(4′maleimidylphenyl)-4-methyloumarin (CPM). CPM becomes fluorescent upon covalent conjugation (Parvari et al, Anal. Biochem. 133:450-456 (1983)). The reactions were carried out in parallel under typical conditions used for labeling proteins, measuring the increase in fluorescence upon formation of the conjugate (Table 1). Cysteine-free MBP_{wt} shows very slight reactivity, presumably due to reaction with surface lysines, since maleimides react slowly with primary amines as well as thiols (Hermanson, Bioconjugate Techniques, 1 ed. Academic Press, San Diego, pp. 148 (1996)). The metal- and oxidatively-protected thiols in MBP_{wt}::ZifQNK•Zn and MBP_{wt}::ZifQNKₒₓ react with CPM at the same very slow rate as detected for the thiol-free protein. The unprotected thiols in MBP₁₄₁, and MBP_{wt}::ZifQNK_{red} react 10,000-fold more rapidly than the protected thiols, with the reaction being >95% complete in 10 or 30 minutes respectively. Both metal- and redox-mediated strategies therefore provide excellent protection and are readily reversible.

TABLE 1
Reaction rates for the conjugation of 7-diethylamino-3-
(4′maleimidylphenyl)-4-methyloumarin (CPM) to
protected and deprotected cysteines

| Protein | t_{1/2} (min) |
|---|---|
| MBP_{wt} | 31,100 |
| MBP_{141C} | 2.5 |
| MBP_{wt}::ZifQNK•Zn | 27,500 |
| MBP_{wt}::ZifQNKₒₓ | 28,800 |
| MBP_{wt}::ZifQNK_{red} | 5.8 |

### Double Labeling

To investigate site-specific labeling with two different fluorophores, C-terminal ZifQNK fusions with MBP₁₄₁ were constructed with a thrombin-cleavable peptide linker (MBP₁₄₁::tb::ZifQNK). Cy5 maleimide mono-reactive dye and tetramethylrhodamine-5-maleimide (TMR) were used as the fluorescent labels. Both the metal- and redox-mediated protection strategies were used to generate the two possible labeling combinations (i.e., a total of four experiments): first attachment of Cy5 to the unprotected Cys141, followed by deprotection (chelation or reduction) and attachment of two TMR labels to ZifQNK (MBP₁₄₁(Cy5)::tb::ZifQNK(TMR)₂; and addition of label in the reverse order to generate MBP₁₄₁(TMR)::tb::ZifQNK(Cy5)₂.

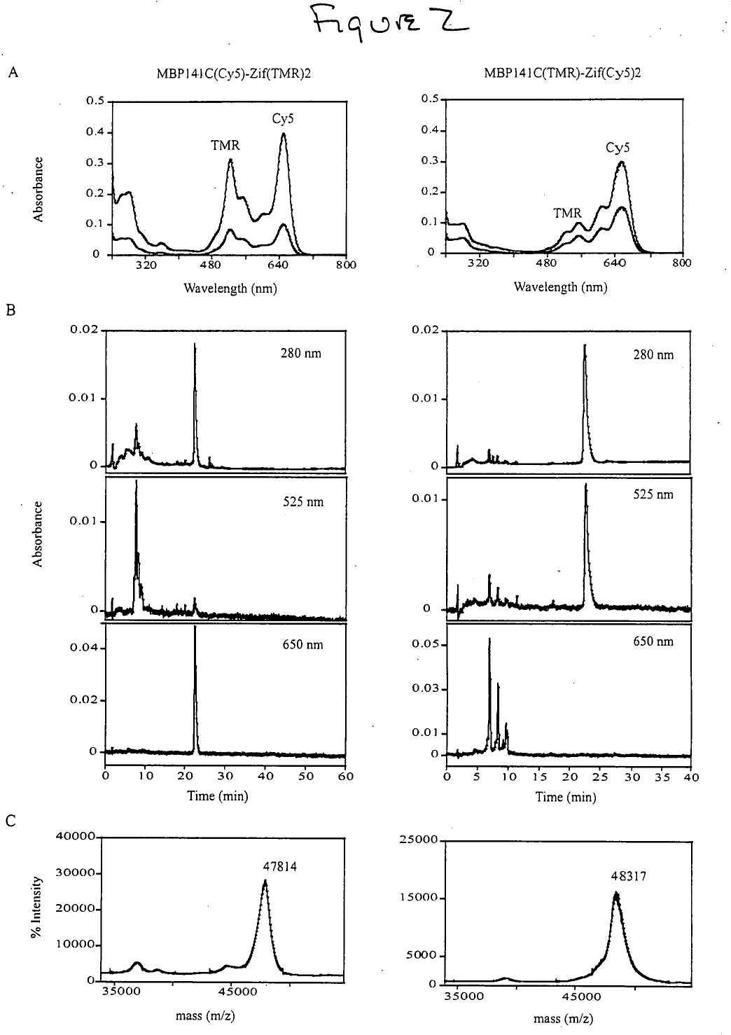

After the first reaction, the protein:fluorophore ratio was determined by absorbance spectroscopy, and was found to be approximately 1:1 in all four cases, consistent with complete reaction of the unprotected thiol in MBP₁₄₁, and full protection of the two thiols in the ZifQNKₒₓ or ZifQNK-Zn²⁺ domain. In the second reaction, the ZifQNK was first deprotected by addition of chelator or reductant, and reacted with the other fluorophore. The stoichiometry of the reaction was determined by absorbance spectroscopy and mass spectrometry (FIG. 2, Table 2). In all four cases, the ratios were 1:1:2 for protein:fluorophore #1:fluorophore #2, consistent with the expected labeling pattern. The masses were also as expected for the appropriately labeled protein (Table 2). The labeled MBP₁₄₁ and ZifQNK domains were separated by thrombin cleavage of the linker peptide to determine the degree of mislabeling (first fluorophore on ZifQNK; second fluorophore on MBP₁₄₁) by the optical absorbance and retention times of the fragments (FIG. 2). In all four cases, no evidence of mislabeling was observed. Taken together, these results are therefore consistent with the intended, site-specific, double labeling patterns, and show that both redox- and metal-mediated reversible thiol protection strategies work well with ZifQNK.

TABLE 2
Masses of modified proteins and peptide fragments

| | Theo- | Experi- |
|---|---|---|
| | retical | mental |
| | mass^{a} | mass^{b} |
| Polypeptide | (Da) | (Da) |
| MBP_{wt}::ZifQNK | 46213 | 46200 |
| MBP₁₄₁(TMR)::th::ZifQNK(Cy5)₂ | 48250 | 48317 |
| MBP₁₄₁(TMR)^{c} | 41820 | 41873 |
| ZifQNK(Cy5)^{c} | 5657 | 5663 |
| ZifQNK(Cy5)₂^{c} | 6435 | 6446 |
| MBP₁₄₁(Cy5)::th::ZifQNK(TMR)₂ | 47953 | 47814 |
| MBP₁₄₁(Cy5)^{c} | 42117 | 42157 |
| ZifQNK(TMR)^{c} | 5360 | 5386 |
| ZifQNK(TMR)₂^{c} | 5842 | 5866 |
| βZif(IAF)₂::th::MBP₁₄₁(TMR)::th::ZifQNK(Cy5)₂ | 51417 | 51578 |

^{a}Theoretical masses calculated using DNA Strider version 1.2.
^{b}Experimental masses measured using MALDI-TOF mass spectrometer as described.
^{c}Peptide fragments obtained by thrombin cleavage

### FRET in Doubly-Labeled Proteins

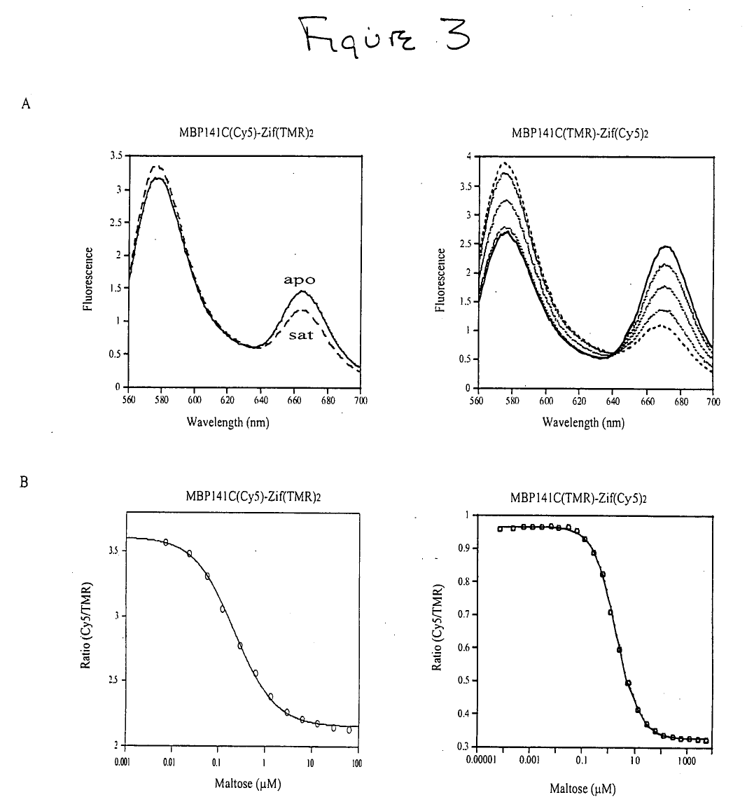

Both types of doubly-labeled protein exhibited a maltose-dependent decrease in FRET between the TMR donor and Cy5 acceptor fluorophores (FIG. 3). The distances between the attached fluorophores is expected to be less in the ligand-bound closed conformation than in the open conformation of the apo-protein. It is therefore likely that orientation- rather than distant-dependent effects dominate the FRET mechanism in this system (Lakowicz, Principles of Fluorescence Spectroscopy, 2^{nd} ed. kluwer Academic Plenum Publishers, New York, pp. 419 (1999)). Furthermore, the magnitude of the change differs in the two constructs: MBP₁₄₁(TMR)::tb::ZifQNK(Cy5)₂ shows a 3-fold change in the ratio of the donor:acceptor emission intensities upon addition of maltose, whereas MBP₁₄₁(Cy5)::tb::ZifQNK(TMR)₂ shows only a 0.1-fold change. The maltose affinities of the labeled and unlabeled proteins are similar (FIG. 3), indicating that the two fluorophores did not significantly perturb the conversion between the open and closed conformations.

### Triple Labeling

To investigate labeling with three different fluorophores, a MBP141 was constructed with βZif fused to the N-terminus, and ZifQNK to the C-terminus, using a thrombin-cleavable linker peptide in each case (βZif::tb::MBP₁₄₁::tb::ZifQNK). βZif and ZifQNK form an orthogonally protected pair: redox-mediated protection has to be used for βZif, mandating the metal-mediated strategy for ZifQNK in this case. The order in which modifications and deprotections are carried out is important: first, the unreacted thiol is modified; second, βZifₒₓ is deprotected by reduction, and modified; third, ZifQNK•Zn²⁺ is deprotected by chelation, and modified. Steps two and three cannot be inverted, because deprotection of ZifQNK•Zn²⁺ requires addition of reductant, which would also deprotect βZifₒₓ.

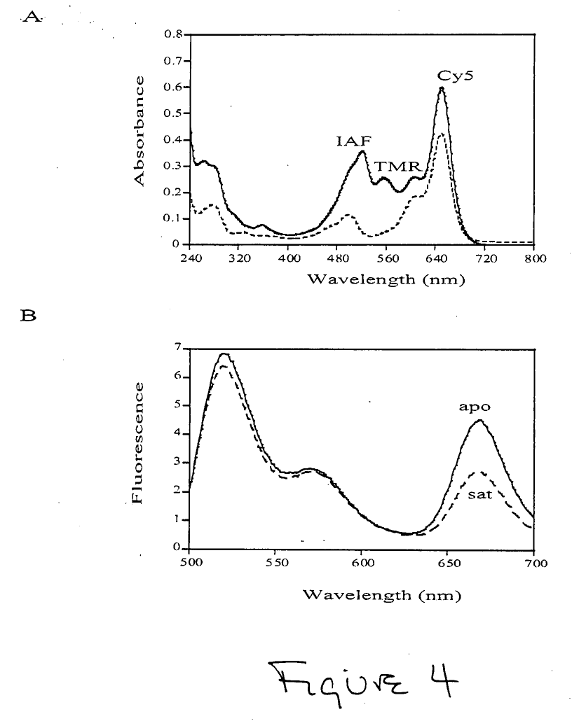

Cy5, TMR and 5-iodoacetamide fluoroscein (IAF) were used as the labels. Two proteins with different labeling patterns were prepared using the appropriate order of modification and deprotection steps: βZif(IAF)₂::tb::MPB₁₄₁(Cy5)::tb::ZifQNK(TMR)₂ and βZif(IAF)₂::tb::MPB₁₄₁(TMR)::tb::ZifQNK(Cy5)₂. Labeling stoichiometries were determined by absorbance spectroscopy for the single and double modifications, but not for the triply labeled proteins, due to the spectral overlap of TMR and IAF (FIG. 4a). The stoichiometry was also confirmed by measuring the mass of triple modified protein (Table 2). The degree of mislabeling was determined by cleaving both N- and C-terminal fusions with thrombin and separating the labeled products on HPLC (data not shown). The unprotected cysteine and the ZifQNK cysteines were exclusively modified with the correct fluorophores. The βZif cysteines were correctly labeled with at least one IAF. The IAF reaction did not quite reach completion (˜90%), however, leaving the second cysteine in some of the βZif fusions free to react with the fluorophore in the third modification.

### FRET in Triply-Labeled Proteins

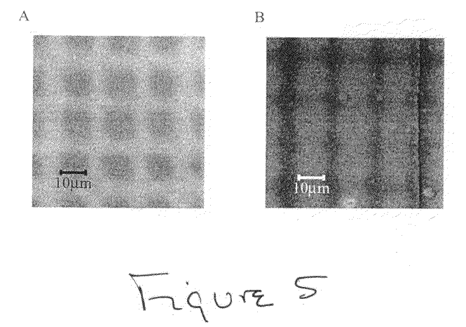

IAF/TMR and TMR/Cy5 both constitute FRET pairs. It is therefore possible to construct an intramolecular FRET relay where excitation energy can be transferred from IAF to Cy5 via TMR (FIG. 5). As predicted, βZif(IAF)₂::tb::MPB₁₄₁ (Cy5)::tb::ZifQNK(TMR)₂ demonstrated a complete FRET relay but βZif(IAF)₂::tb::MPB₁₄₁(TMR)::tb::ZifQNK(Cy5)₂ did not, presumably because the separation between IAF and TMR is within the Förster distance in βZif(IAF)₂::tb::MPB₁₄₁(Cy5)::tb::ZifQNK(TMR)₂ (42 Å) but exceeds the Förster distance in βZif(IAF)₂::tb::MPB₁₄₁(TMR)::tb::ZifQNK(Cy5)₂ (61 Å). FRET between TMR and Cy5 still occurs in βZif(IAF)₂::tb::MPB₁₄₁(TMR)::tb::ZifQNK(Cy5)₂ when TMR is excited (50 Å). The FRET relay demonstrated a maltose-dependent decrease (FIG. 4B).

### Protein Immobilization

GBP₁₄₉::ZifQNKₒₓ was derivatized with Cy5 at Cys149. The disulfide was reduced and GBP₁₄₉(Cy5)::ZifQNK_{red} was reacted with a glass slide patterned with bis-maleimidoethane (BMOE) (FIG. 5A). The BMOE pattern was generated by protecting thiol silane from photooxidation with a 10 μm beehive mask as described above. An image of a slide prepared with a Cy5-modified GBP lacking the ZifQNK fusion was also taken (FIG. 5B). As can be seen, the GBP₁₄₉(Cy5)::ZifQNK gave the expected square grid pattern corresponding to reaction with the maleimide, whereas the pattern produced by the control protein was significantly dimmer, and is consistent with physisorption of the protein in the irradiated squares where there is a preponderance of negatively charged groups resulting from photooxidation (Bhatia et al, J. Am. Chem. Soc. 114:4432-4433 (1992)).

Summarizing, the foregoing studies demonstrate that fusions with one or two zinc finger derivatives allow two or three sites to be modified independently by reversible thiol protection schemes that exploit metal coordination or disulfide formation. Both methods produce orthogonal protein modifications with no apparent mislabeling. Both MBP₁₄₁(TMR)::th::ZifQNK(Cy5)₂ and MBP₁₄₁(Cy5)::th::ZifQNK(TMR)₂ were rapidly produced by simply switching the order of reactants, unlike many competing methods which require additional synthesis steps (Hofmann and Muir, Curr. Opin. Biotechnol. 13:297-303 (2002), Zhang et al, Biochemistry 42:6735-6746 (2003)). Both labeling combinations resulted in ligand-induced FRET decreases. MBP₁₄₁(TMR)::th::ZifQNK(Cy5)₂, in particular, generated a larger ligand-mediated signal change than any previously reported intramolecular FRET biosensor (Hofmann et al, Bioorg. Med. Chem. Lett. 11: 3091-3094 (2001), Fehr et al, Proc Natl Acad Sci USA 99: 9846-9851 (2002), Fehr et al, Curr Opin Plant Biol 7: 345-351 (2004), Lager et al, FEBS Lett. 553:85-89 (2003)). The large FRET change cannot be explained in terms of distance dependent effects because the distance change is too small and because the separation between fluorophores gets smaller upon ligand binding which should produce an increase rather than a decrease in FRET. Instead, it is proposed that the observed FRET change is due to an orientation effect (Lakowicz, Principles of Fluorescence Spectroscopy, 2^{nd} ed. Kluwer Academic Plenum Publishers, New York, pp. 419 (1999)). The 2:1 ratio of fluorophores did not appear to interfere with FRET or correct immobilization.

All documents and other information sources cited above are hereby incorporated in their entirety by reference.

What is claimed is:

1. A method of site specifically labeling a protein comprising:
i) constructing a fusion protein comprising:
(a) a protein comprising at least a single unprotected and uniquely reactive amino acid, and
(b) at least one domain comprising one or more uniquely protected reactive amino acids,
ii) reacting said unprotected amino acid of protein (a) with a first modifying agent so that a covalent linkage between said unprotected amino acid and said first modifying agent is formed, and
iii) deprotecting said one or more reactive amino acids of domain (b) and reacting said one or more deprotected reactive amino acids with a second modifying agent so that a covalent linkage between said one or more deprotected reactive amino acids and said second modifying agent is formed.


SEQUENCE LISTING (9 sequences)

SEQ ID NO: 1 - protein, 47 aa, Artificial Sequence
  Description of Artificial Sequence Synthetic peptide
GLVPRGSTGEKPYKCPECGKSFSRSDHLSRHQRTHQXKKGSHHHHHH

SEQ ID NO: 2 - protein, 27 aa, Artificial Sequence
  Description of Artificial Sequence Synthetic peptide
MTGEKPYKCPECGKSFSRSLVPRGSGG

SEQ ID NO: 3 - DNA, 69 bp, Artificial Sequence
  Description of Artificial Sequence Synthetic oligonucleotide
ggaggttcaacaggtgagaaaccgtacaagtgcccggagtgtggcaaatcattctctcgatcggaccat

SEQ ID NO: 4 - DNA, 69 bp, Artificial Sequence
  Description of Artificial Sequence Synthetic oligonucleotide
cgggatcctatcacttcttgttctgatgtgtccgttggtgacgggatagatggtccgatcgagagaatg

SEQ ID NO: 5 - DNA, 36 bp, Artificial Sequence
  Description of Artificial Sequence Synthetic oligonucleotide
ctcacctgttgaacctcccttggtcagcttagtctg

SEQ ID NO: 6 - DNA, 49 bp, Artificial Sequence
  Description of Artificial Sequence Synthetic oligonucleotide
ggaattccatatgacaggtgagaaaccgtacaagtgcccggagtgtggc

SEQ ID NO: 7 - DNA, 71 bp, Artificial Sequence
  Description of Artificial Sequence Synthetic oligonucleotide
ccttcttcgattttgcccccggatcctcgagggacgagcgatcgagagaatgatttgccacactccgggca

SEQ ID NO: 8 - DNA, 26 bp, Artificial Sequence
  Description of Artificial Sequence Synthetic oligonucleotide
gaactgaatgcaaaggtaagagcgcg

SEQ ID NO: 9 - DNA, 27 bp, Artificial Sequence
  Description of Artificial Sequence Synthetic oligonucleotide
cgcgctcttacctttgcatttcagttc